(19) United States
(12) Patent Application Publication
(10) Pub. No.: US 2025/0025394 A1
(43) Pub. Date: Jan. 23, 2025

(54) SEMI-PERMANENT SMOOTHING HAIR COMPOSITION AND USES THEREOF

(71) Applicant: HB SCIENCE, LLC, Agoura Hills, CA (US)

(72) Inventor: Daniel MOSHIRIAN, Agoura Hills, CA (US)

(73) Assignee: HB SCIENCE, LLC, Agoura Hills, CA (US)

(21) Appl. No.: 18/901,693

(22) Filed: Sep. 30, 2024

Related U.S. Application Data
(63) Continuation of application No. PCT/US2023/083824, filed on Dec. 13, 2023.
(60) Provisional application No. 63/387,181, filed on Dec. 13, 2022.

(51) Int. Cl. A61K 8/43; A45D 7/04; A45D 19/00; A61K 8/19; A61K 8/898; A61Q 5/06

(52) U.S. Cl. CPC A61K 8/43; A45D 7/04; A45D 19/005; A61K 8/19; A61K 8/898; A61Q 5/06; A61K 2800/884

(57) ABSTRACT

Provided herein are catalyst cream base compositions and activator compositions, kits comprising the same and methods of use thereof. The catalyst cream base composition can comprise calcium hydroxide and a silicone blocker and the activator compositions can comprise guanidine hydrochloride. The compositions can be used for the preparation of smoothing compositions. The methods of use can comprise methods of semi-permanently smoothing hair. The kits can comprise catalyst cream base compositions and activator compositions and instructions to use the same for semi-permanently smoothing hair.

### CROSS-REFERENCE TO RELATED APPLICATIONS

This application is a continuation of International Patent Application No. PCT/US2023/083824, filed Dec. 13, 2023, which claims the benefit of priority to U.S. Provisional Appl. Ser. No. 63/387,181, filed Dec. 13, 2022, the contents of which are incorporated by reference herein in their entirety.

### FIELD OF THE DISCLOSURE

The present invention relates generally to smoothing hair compositions, and more specifically to methods of the use of calcium hydroxide and activators such as guanidine hydrochloride and guanidine carbonate to semi-permanently smooth hair.

### BACKGROUND

Hair relaxers, straighteners, or keratin smoothing treatments use chemicals such as thio (ammonium thioglycolate), lye (sodium hydroxide), and formaldehyde to reduce or remove curls.

The existing compositions and methods are usually permanent and generally cannot be reapplied on previously treated “relaxed” hair or can be semi-permanent (temporary) but can release formaldehyde during application.

There is still an unmet need in the art for non-permanent or semi-permanent smoothing hair compositions that do not release formaldehyde.

### SUMMARY

Provided herein are catalyst cream base compositions comprising calcium hydroxide and activator compositions comprising guanidine hydrochloride used for the preparation of smoothing compositions, and methods of use thereof, including methods of semi-permanently smoothing hair.

An embodiment provides a method of semi-permanently smoothing hair.

The method can comprise a) contacting hair with a smoothing composition comprising calcium hydroxide, guanidine hydrochloride and silicone blockers, b) roughly drying the hair, and c) blow-drying the hair to 100% dryness with tension thereby semi-permanently smoothing the hair. Contacting can comprise contacting hair with a smoothing composition for about ≤45 minutes. The tension can be created with a ceramic round brush or boar bristle brush. Contacting the hair with the smoothing composition can comprise applying the smoothing composition from scalp or off scalp to ends in sub-sections and saturating the hair from scalp or off scalp to ends. In an alternative embodiment, the drying step can comprise towel drying hair rather than blow drying hair to 100%.

The method can further comprise, prior to contacting the hair with the smoothing composition, shampooing the hair with a cleansing shampoo. The method can further comprise flat ironing the hair after the blow drying. The method can further comprise rinsing the hair, washing the hair with a demineralizing cleansing shampoo, and conditioning the hair with a conditioner. The method can further comprise towel drying the hair and applying a hair or laminator type serum to the hair. The smoothing composition can comprise a catalyst cream base comprising calcium hydroxide and a silicone blocker; and an activator composition comprising guanidine hydrochloride and/or guanidine carbonate. The catalyst cream base and the activator can be mixed prior to use. The catalyst cream base can further comprise behentrimonium chloride, cetrimonium chloride and/or stearalkonium chloride, quaternium-91, one or more emulsifiers, guar hydroxypropyltrimonium chloride derivatives, one or more conditioning agents, mineral oil, one or more esters, and a solvent.

In a preferred embodiment, the method can comprise a) washing hair with a demineralizing cleansing shampoo, b) towel drying the hair, c) contacting the hair with a smoothing composition comprising calcium hydroxide, guanidine hydrochloride and silicone type blockers, d) contacting the hair with a smoothing composition for about ≤45 minutes, e) shampooing the hair using demineralizing cleansing shampoo, f) towel drying the hair, g) contacting hair with laminator type serum for about ≤15 minutes, h) rinsing the hair with water, i) blow-drying the hair with a round brush to 100% dryness with tension thereby semi-permanently smoothing the hair, and j) flat ironing the hair after blow-drying. Contacting the hair with the smoothing composition can comprise applying the smoothing composition from scalp or off scalp to ends in sub-sections and saturating the hair from scalp or off scalp to ends. In an alternative embodiment, the drying step can comprise towel drying hair rather than blow drying hair to 100%.

The activator can further comprise a solvent, one or more preservatives, one or more conditioning gums, and one or more buffering solutions. The cleansing shampoo can comprise about 5%-60% primary and secondary surfactants, about 0.01%-7.0% chelating agent, about 0.01%-5.0% highly charged anionic polymer, and about 0.001%-5.0% buffering agent. The cleansing shampoo can further comprise about 30%-85% solvent and 0.01%-3.0% preservatives, wherein optionally the solvent can be water. The catalyst cream base can comprise about 0.10%-10% calcium hydroxide.

The catalyst cream base can comprise about 0.01 to about 4.5% calcium hydroxide. The catalyst cream base can comprise about 0.10%-5.0% behentrimonium chloride, cetrimonium chloride and/or stearalkonium chloride, about 0.1%-10% emulsifiers, about 0.1%-10.0% quaternium-91, about 0.01%-3.0% guar hydroxypropyltrimonium chloride derivatives, about 0.1%-8.0% conditioning agents, about 0.01%-20.0% silicones, about 0.10%-10.0% mineral oil, about 0.01%-20% esters, and a solvent, wherein optionally the solvent can be water. The silicone blocker can be liquid amino phenyl silicone resin (i.e., aminopropyl phenyl trimethicone).

The activator composition can comprise about 5%-50% guanidine hydrochloride. The activator composition can comprise about 5%-13% guanidine hydrochloride. The activator composition can comprise about 35%-50% guanidine hydrochloride. The activator composition can comprise about 30%-85% solvent, about 0.10%-4% preservatives, about 0.05%-5% conditioning gums, and about 0.01%-7% buffering solutions, wherein optionally the solvent is water. The activator composition can further comprise a blend of active ingredients with cross-linking action based on the synergistic association of unsaturated dicarboxylic acid (itaconic acid), amino acid (arginine), pro-vitamin B5 (D-panthenol), and polysaccharides extracted from linseed and chia seeds, acting through repair and protection mechanisms and proactive treatment of chemically damaged hair. In an alternative embodiment, guanidine hydrochloride can be substituted or combined with a guanidine carbonate activator. The hair serum can comprise about 20%-90% solvent, about 0.01%-3% guar hydroxypropyltrimonium chloride, about 0.01%-5% hydroxyethylcellulose, and about 0.10%-8% amodimethicone, optionally wherein the solvent can be water. The hair system can be substituted with a laminator type serum. The hair serum or laminator type serum can be applied after rinsing the hair. The smoothing composition can generate water soluble calcium chloride and not generate water insoluble calcium carbonate. The smoothing composition can temporarily manipulate, weaken, stretch and bend the bonds within the hair's cortex into a straighter position and remove excess curl and reduce frizz. The methods can be repeated to previously treated hair at, for example 8 weeks after an initial application.

An embodiment provides a kit comprising: a) a catalyst cream base comprising about 0.10%-10% calcium hydroxide, about 0.10%-5.0% behentrimonium chloride, cetrimonium chloride and/or stearalkonium chloride, about 0.1%-10% emulsifiers, about 0.1%-10.0% quaternium-91, about 0.01%-3.0% guar hydroxypropyltrimonium chloride derivatives, about 0.1%-8.0% conditioning agents, about 0.01%-20.0% silicones, about 0.10%-10.0% mineral oil, about 0.01%-20% esters, and a solvent, wherein optionally the solvent is water, and b) an activator composition comprising about 5%-50% guanidine hydrochloride, about 30%-85% solvent, about 0.10%-4% preservatives, about 0.05%-5% conditioning gums, and about 0.01%-7% buffering solutions, wherein optionally the solvent can be water. The kit can further comprise of chelating cleanser to remove build-up or any other residue left on hair, smoothing emulsion containing calcium hydroxide, and laminator with a blend of different carboxylic acids. In an alternative embodiment, guanidine hydrochloride can be substituted or combined with a guanidine carbonate activator.

The activator composition can comprise about 35%-50% guanidine hydrochloride. The activator composition can comprise about 5%-13% guanidine hydrochloride. The catalyst cream base can comprise about 0.01% to about 4.5% calcium hydroxide. The silicone blocker can be liquid amino phenyl silicone resin. The activator composition can further comprise a blend of active ingredients with cross-linking action based on the synergistic association of unsaturated dicarboxylic acid (itaconic acid), amino acid (arginine), pro-vitamin B5 (D-panthenol), and polysaccharides extracted from linseed and chia seeds, acting through repair and protection mechanisms and proactive treatment of chemically damaged hair. In an alternative embodiment, guanidine hydrochloride can be substituted or combined with a guanidine carbonate activator.

The laminator type serum composition can comprise various emulsifiers, fatty alcohols, esters, humectants, non-ionic rheology modifiers, bond repair and protective actives, and combinations of carboxylic acids (di or mono or tri) at very low pH.

The demineralizing cleansing shampoo composition can comprise a primary surfactant, a secondary surfactant, a chelating agent, a highly charged anionic polymer, and a buffering agent. The catalyst cream base can comprise (i) guar hydroxypropyltrimonium chloride or a derivative thereof, (ii) behentrimonium chloride, cetrimonium chloride, and stearalkonium chloride, quaternium-91, and (iii) calcium hydroxide.

An embodiment provides a kit for smoothing hair comprising a) a demineralizing cleansing shampoo composition, b) a catalyst cream base comprising calcium hydroxide, c) an activator composition comprising guanidine hydrochloride, d) a hair serum or laminator type serum, and e) instructions to smooth hair. The kit can further comprise of chelating cleanser to remove build-up or any other residue left on hair, smoothing emulsion containing calcium hydroxide, and laminator with a blend of different carboxylic acids. The kit can further comprise a silicone blocker. The silicone blocker can be liquid amino phenyl silicone resin. In an alternative embodiment, guanidine hydrochloride can be substituted or combined with a guanidine carbonate activator.

### DETAILED DESCRIPTION

The present disclosure provides catalyst cream base compositions comprising calcium hydroxide and activator compositions comprising guanidine hydrochloride and/or guanidine carbonate, which can be used for the preparation of smoothing compositions, and methods of use thereof. The methods of use include methods of semi-permanently smoothing hair.

### Overview

Hair is made up of keratin, a fibrous structural protein, which contains about 18% cysteine as calculated as part of the overall amino acid composition. A hair fiber has three main regions: the cuticle, cortex, and medulla. The medulla makes up the core of the hair fiber and can be partially or entirely absent in fine hair fibers. The cortex is the major bulk of the hair fiber and is made up of microfibrils of intermediate filaments, which are in turn composed of α-keratins in α-helical form. The individual filaments of cortical cells are separated by a cell membrane complex of keratin associated proteins (KAPs), which have a high cysteine content. Keratin α-helices are coiled by ionic forces, hydrogen bonds, van der Waals forces, and disulfide bonds. The cuticle, enclosing the hair cortex, is made up of superimposed layers of cells in a scaled structure. Each cuticle cell contains sub-lamellar layers (epicuticle, A-layer, exocuticle, and endocuticle) with proteins cross-linked by disulfide bonds.

The presence of disulfide bonds in cysteine residues of the hair keratin protein determine the maintenance of the shape of the hair, which can be altered with chemicals. Harsh chemicals, however, can damage the hair fiber, reduce the cross-linking density, and decrease the hair's physio-mechanical properties. Excessive or repeated hair chemical treatments can produce irreversible changes in hair texture and can result in a fracture of the hair fiber once the cuticle is removed.

Hair relaxers, straighteners or keratin smoothing treatments use chemicals such as thio (ammonium thioglycolate), lye (sodium hydroxide) and no-lye relaxers (guanidine hydroxide, lithium hydroxide, potassium hydroxide), formaldehyde, and non-formaldehyde (formaldehyde releasing agents) to reduce or remove frizz and curls.

Formaldehyde is different from the other ingredients because formaldehyde is not the hair straightening active ingredient. The hair is remodeled straight because water breaks hydrogen bonds of the keratin molecule as happens in a regular blow-dry. The newly redesigned keratin is then kept in this shape because formaldehyde crosslinks the keratin filaments in such a perfect alignment that the hair is set straight and shines like no virgin straight hair is capable of. The light that strikes the hair reflects from the realigned keratin filaments and brings the effect of a brighter shiny hair. The analysis of the reactivity of formaldehyde and wool keratin shows that formaldehyde forms cross-links with the keratin amino acids, arginine, lysine, tyrosine, histidine, and the amide derivatives of aspartate and glutamate.

Hair relaxers use chemicals to straighten hair by breaking the disulfide bonds found in the cortex layer of hair and re-forming them.

Hair relaxers work by reducing the cystine to cysteine bonds and allowing the hair structure to be changed as follow:

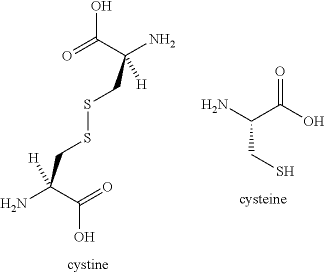 [SMILES: N[C@@H](CSSC[C@H](N)C(=O)O)C(=O)O ; N[C@@H](CS)C(=O)O]

Cystine (left) is reduced to form cysteine (right).

There are two main types of hair relaxers: lye and no-lye. The lye hair relaxers contain sodium hydroxide (NaOH) and have an extremely high pH (in the range 12-14). These types of hair relaxers break bonds and strip the hair of its natural oils. Heating is also often used, and this damages the hair even more.

Due to increasing concerns about the damage caused by lye hair relaxers, companies have produced no-lye hair relaxers. These still contain basic compounds and are still caustic, but their pH is not as high. These products either use calcium hydroxide and guanidine carbonate or lithium hydroxide. These are bases and act in a similar manner to sodium hydroxide.

No-Lye relaxers are gentler on scalp, but they need to be left on scalp longer leading to drying of hair due to calcium build-up. Guanidine carbonate is a strong alkaline, organic salt utilized primarily as an ingredient in hair straightening and depilatory systems. When combined with calcium hydroxide in (no-lye) hair relaxers it reacts to form a less irritating system than sodium hydroxide-based (lye) systems. It also is used as a buffering agent and a pH adjuster.

When using guanidine hydrochloride as detailed herein to react with calcium hydroxide relaxer, the resulting product is calcium chloride (a salt). Calcium chloride is water soluble, therefore there is a significantly less drying effect on the hair as compared to calcium carbonate.

Guanidine hydrochloride is a well-known substance listed in the Merck Index. It is not known for use as a hair relaxer.

### Methods of Semi-Permanently Smoothing Hair

Provided herein are methods of semi-permanently smoothing hair without excess drying.

The methods described herein are for semi-permanently smoothing hair. By semi-permanently smoothing hair, it is meant that the method can temporarily restructure the keratin fibers comprised in the hair. “Semi-permanent smoothing” of hair refers to any non-permanent treatment of hair, as opposed to a permanent relaxing method. While “relaxing” can refer to a permanent modification of hair, smoothing methods, as described herein can last for a period of time, and slowly wash out of the hair, at which point a user can optionally decide to reapply the treatment to achieve the result again. Because the smoothing method is semi-permanent, there is little risk of damaging the hair by reapplying the smoothing treatment after a period of time. The treatment is “semi-permanent”, which is meant to include that the treatment is to be understood as “temporary”, “demi permanent”, “short-term”, “momentary”, “transitory”, “ephemeral” and the like.

The methods described herein temporarily manipulate, weaken, stretch and bend the bonds within the hair's cortex into a straighter position and remove excess curl and frizz without breaking these bonds. This allows for the hair to revert back to its natural state after a certain period of time. The term “bonds” as used herein can refer to any bond that may exist within the hair's cortex, and may include for example hydrogen, ionic and disulfide bonds in the hair. All such bonds may be impacted and altered by the methods described herein, but in such a minimal way that the methods are non-permanent, as the impacting/altering of the bonds are reversible, which allows the methods to be repeated over time without damaging the hair.

In sharp contrast, a permanent relaxing treatment does not wash out, and a user needs to wait for new untreated hair to grow out prior to being able to re-apply the relaxing treatment. That is, a permanent relaxing treatment cannot be applied to hair that has already been treated once with a relaxing treatment.

Cysteines that never realign do not reform disulfide bonds, which are permanently broken. That is there is no point of return, and regardless of the amount of time after the treatment, the cysteines will not be able to realign and generate disulfide bonds again or not a sufficient number of cysteines will be able to realign and generate disulfide bonds again. The only way to regain curl and movement in the hair is by outgrowing the portion of hair that was treated with the relaxing treatment. Furthermore, the hair cannot be treated with a relaxing treatment again or the hair is at risk of breaking off.

A smoothing composition can provide a non-permanent treatment to the hair. Such treatment is less damaging to the hair and can be re-applied as necessary.

Smoothing hair means that, after a certain amount of time (as discussed below) a greater amount of cysteines will be able to realign and generate disulfide bonds again: the curl reduction is not permanent, the hair is smoothened. In such a case, the curl and movement in the hair can be obtained simply by waiting for the product to wash out. The semi-permanent smoothing treatment is temporary. The effect can last for about 12-14 weeks (i.e., about 10, 11, 12, 13, 14, 15 or 16 weeks) without needing any specific additional treatment of the hair. Once the effect washes off, a user may decide to reapply the semi-permanent smoothing treatment to regain 12-14 weeks of smoothing effect.

The method can comprise a) contacting hair with a smoothing composition comprising calcium hydroxide and guanidine hydrochloride, b) roughly drying the hair, and c) blow-drying the hair to 100% dryness with tension thereby semi-permanently smoothing the hair. In an alternative embodiment, the drying step can comprise towel drying hair rather than blow drying hair to 100%.

In a preferred example, the method can comprise a) washing hair with a demineralizing cleansing shampoo, b) towel drying the hair, c) contacting the hair with a smoothing composition comprising calcium hydroxide, guanidine hydrochloride and silicone type blockers, d) contacting the hair with a smoothing composition for about ≤45 minutes, e) shampooing the hair using demineralizing cleansing shampoo, f) towel drying the hair, g) contacting hair with laminator type serum for about ≤15 minutes, h) rinsing the hair with water, i) blow-drying the hair with a round brush to 100% dryness with tension thereby semi-permanently smoothing the hair, and j) flat ironing the hair after blow-drying. In an alternative embodiment, the drying step can comprise towel drying hair rather than blow drying hair to 100%.

By “contacting” hair with a smoothing composition, it is meant that a smoothing composition is applied to the hair. For example, contacting hair with a smoothing composition can comprise applying a smoothing composition from scalp or off scalp to ends in sub-sections and saturating hair from scalp to ends. By “off scalp,” it is meant applying near or adjacent to the scalp, preferably between about 0.25 to about 0.5 inches off of the scalp.

Contacting can comprise contacting hair with a smoothing composition for about ≤45 minutes. For example, a smoothing composition can be applied for about 5, 10, 15, 20, 25, 30, 35, 40, 45 or 50 minutes. The length of time can be evaluated based on the texture of the hair, or as evaluated by a professional applying a smoothing composition.

The methods are semi-permanent and can be repeated in about 6, 7, 8, 9, 10, 11, or 12 weeks after an initial application and as necessary to maintain the smoothing effect on the hair. The interval of time in between two applications can be determined by a user and/or a professional hairdresser to ensure that a desired effect is obtained and maintained. The methods described herein are reversible. By “reversible” it is meant that after an initial application of the composition described in the instant methods, the hair is modified, but such modification can be reverted, and the hair can return to its initial state after a certain period of time. Without wishing to be bound to any specific length, it was observed that after application of the smoothing composition described here, following the instant methods, the hair can revert back to its natural state after about 12-14 weeks. The reversion being a progressive process, the methods can be repeated prior to the return to the initial state, when the hair is in an intermediate state (i.e., not the state initially obtained after the application of the smoothing composition-smoothened hair-, but not the hair in its natural state—non-smoothened hair—).

After application of a smoothing composition, hair can be dried. Drying hair (i.e., removing moisture, which can either be completely −100% dry- or incompletely-less than 100% dry) can be performed in many different ways, from naturally (i.e., air drying) to using props or tools using or not using any tension (i.e., a towel, a blow dryer, alone or in combination with a comb or brush). In the method described herein, the hair can be dried after application of a smoothing composition. The drying can involve a first step during which the hair is incompletely dried without using any tension. For example, the hair can be roughly dried to about 60-70% dryness (e.g., about 50, 55, 60, 65, 70, or 75% dryness). As used herein, “rough drying” is meant to refer to the drying of the hair to a point that is dry enough to round brush the hair. In an example, rough drying is towel drying the hair. When not using tension, the hair can be dried using a towel or a blow dryer, without applying any tension during the use of the blow dryer. The drying can then involve a second step during which the hair is completely dried (e.g., to 100% dryness) using tension. By “using tension”, it is meant that the hair (usually wet, e.g., after washing the hair or rough dried) can be stretched while being dried. The drying can be done by using a blow dryer from a short distance. This method allows straightening of the hair with minimum damage. The stretching, or application of tension can be done or created using one's hand, or with a tool, such as a comb or a brush. The tension can be created with, e.g., a ceramic round brush or a boar bristle brush.

Contacting the hair with a smoothing composition can comprise applying a smoothing composition from scalp or off scalp to ends in sub-sections and saturating the hair from scalp or off scalp to ends.

The method can further comprise, prior to contacting the hair with a smoothing composition, shampooing the hair with a demineralizing cleansing shampoo.

Shampooing hair with a demineralizing cleansing shampoo prior to applying a smoothing composition can be used to remove any deposits from the hair to ensure proper contact between the hair surface and a smoothing composition, such that the efficacy of a smoothing composition is optimal. Usually, after shampooing the hair, excess moisture can be removed from the hair by drying the hair with a towel.

That is, the method can comprise (i) shampooing hair with a demineralizing cleansing shampoo, (ii) removing excess moisture by drying the hair with a towel, and (iii) contacting hair with a smoothing composition comprising calcium hydroxide and guanidine hydrochloride.

In an example, the method can comprise further rinsing and shampooing with the demineralizing cleansing shampoo.

In another example, the method can further comprise flat ironing the hair. For example, after application of a smoothing composition, the hair can be dried using a blow dryer, and then flat ironed to shape the hair in a smooth form (i.e., into a straight form).

In another example, the method can comprise contacting hair with a smoothing composition comprising calcium hydroxide and guanidine hydrochloride.

Alternatively, when the application of a smoothing composition is preceded by a cleaning of the hair, the method can comprise (i) shampooing hair with a cleansing shampoo, (ii) removing excess moisture by drying the hair with a towel, and (iii) contacting hair with a smoothing composition comprising calcium hydroxide and guanidine hydrochloride.

The smoothing composition can generate water soluble calcium chloride and not generate water insoluble calcium carbonate such that the hair fibers are not subjected to excess drying. That is, the smoothing composition does not induce dry hair. By “dry hair”, it is meant that contrary to a relaxing composition, the smoothing composition of the instant disclosure does not reduce the amount of moisture and oil present in the hair fiber. Hair that has reduced moisture and oil content (i.e., dry hair) cannot maintain its normal sheen and texture. Such a condition can be induced by a variety of cause, including for example excessive hair washing, or using harsh soaps, alcohols or hair products (such as relaxing treatments), excessive blow-drying, dry air due to the climate, malnutrition, anorexia underactive parathyroid (hypoparathyroidism), underactive thyroid (hypothyroidism), other hormone abnormalities, and Menkes kinky hair syndrome.

A chemical reaction between calcium hydroxide present in a catalyst cream base and guanidine hydrochloride present in an activator composition (i.e., the components of the smoothing composition described herein) can generate calcium chloride. Contrarily to other straightening composition that can generate water insoluble calcium carbonate, the present method can generate calcium chloride, which is a soluble compound. Therefore, the smoothing composition can have a minimal drying effect on the hair. To remove any such soluble deposit from the hair, the hair can be rinsed, washed and optionally conditioned after the application of a smoothing composition. The method can thus further comprise rinsing the hair, washing the hair with a cleansing shampoo, and conditioning the hair with a conditioner.

That is, the method can comprise, in an example, (i) contacting hair with a smoothing composition comprising calcium hydroxide and guanidine hydrochloride and (ii) rinsing and washing the hair with a demineralizing cleansing shampoo, and conditioning the hair with a conditioner, and (iii) applying the laminator type serum to the hair.

Alternatively, when the application of a smoothing composition is preceded by a cleaning of the hair, the method can comprise (i) shampooing hair with a cleansing shampoo, (ii) removing excess moisture by drying the hair with a towel, (iii) contacting hair with a smoothing composition comprising calcium hydroxide and guanidine hydrochloride, (iv) rough drying the hair, (v) blow-drying the hair to 100% dryness, (vi) flat ironing the hair, and (vi) rinsing the hair, washing the hair with a demineralizing cleansing shampoo, and conditioning the hair with a conditioner. In an alternative embodiment, the drying step can comprise towel drying hair rather than blow drying hair to 100%.

The method can further comprise towel drying the hair and applying a hair serum or laminator type serum to the hair. As part of the final shaping/styling of the hair, the hair can be dried using a towel and a hair serum can be applied.

That is the method can comprise (i) contacting hair with a smoothing composition comprising calcium hydroxide and guanidine hydrochloride, (ii) rough drying the hair, (iii) blow-drying the hair to 100% dryness, (iv) flat ironing the hair, (v) rinsing the hair, washing the hair with a demineralizing cleansing shampoo, and conditioning the hair with a conditioner, and (vi) towel drying the hair and applying a hair serum or laminator type serum to the hair. In an alternative embodiment, the drying step can comprise towel drying hair rather than blow drying hair to 100%.

Alternatively, when the application of a smoothing composition is preceded by a cleaning of the hair, the method can comprise (i) shampooing hair with a cleansing shampoo, (ii) removing excess moisture by drying the hair with a towel, (iii) contacting hair with a smoothing composition comprising calcium hydroxide and guanidine hydrochloride, (iv) rough drying the hair, (v) blow-drying the hair to 100% dryness, (vi) flat ironing the hair, (vi) rinsing the hair, washing the hair with a demineralizing cleansing shampoo, and conditioning the hair with a conditioner, and (vii) towel drying the hair and applying a hair serum or laminator type serum to the hair. In an alternative embodiment, the drying step can comprise towel drying hair rather than blow drying hair to 100%.

### Smoothing Composition

A smoothing composition as described herein can comprise a catalyst cream base comprising the calcium hydroxide and a silicone blocker; and an activator composition comprising the guanidine hydrochloride.

The guanidine hydrochloride in the activator composition mixed together with the calcium hydroxide in the catalyst cream based can react with the hair's polypeptide chain of the cortex. Such reaction can temporarily manipulate, weaken, stretch and bend the bonds within the hair's cortex into a straighter position and remove excess curl and frizz without breaking these bonds. It is also envisaged that guanidine hydrochloride can be substituted or combined with a guanidine carbonate activator.

In one aspect, the catalyst cream base can comprise about 0.10%-10% calcium hydroxide. For example, a catalyst cream base can comprise about 0.1, 0.2, 0.5, 0.75, 1, 2, 3, 4, 5, 6, 7, 8, 9, 10, 11, or 12% calcium hydroxide.

In another aspect, the catalyst cream base can comprise about 0.01 to about 4.5% calcium hydroxide. For example, a catalyst cream base can comprise about 0.01, 0.025, 0.05, 0.075, 0.1, 0.2, 0.3, 0.4, 0.5, 0.6, 0.7, 0.8, 0.9, 1, 1.5, 2, 2.5, 3, 3.5, 4, 4.5 or 5% calcium hydroxide.

The catalyst cream base can further comprise behentrimonium chloride, cetrimonium chloride and/or stearalkonium chloride, quaternium-91, one or more emulsifiers, guar hydroxypropyltrimonium chloride derivatives, one or more conditioning agents, mineral oil, one or more esters, and a solvent.

The catalyst cream base can comprise about 0.10%-5.0% behentrimonium chloride, cetrimonium chloride and/or stearalkonium chloride, about 0.1%-10.0% quaternium-91, about 0.1%-10% emulsifiers, about 0.01%-3.0% guar hydroxypropyltrimonium chloride derivatives, about 0.1%-8.0% conditioning agents, about 0.01%-20.0% silicones, about 0.10%-10.0% mineral oil, about 0.01%-20% esters, and a solvent, wherein optionally the solvent can be water.

For example, a catalyst cream base can comprise (i) about 0.1, 0.2, 0.5, 0.75, 1, 1.5, 2, 2.5, 3, 3.5, 4, 4.5, or 5.0% behentrimonium chloride, cetrimonium chloride and/or stearalkonium chloride, (ii) about 0.1, 0.2, 0.5, 0.75, 1, 2, 3, 4, 5, 6, 7, 8, 9 or 10% emulsifiers, quaternium-91, (iii) about 0.01, 0.025, 0.05, 0.075, 0.1, 0.2, 0.3, 0.4, 0.5, 0.6, 0.7, 0.8, 0.9, 1, 1.5, 2, 2.5 or 3.0% guar hydroxypropyltrimonium chloride derivatives, (iv) about 0.1, 0.2, 0.5, 0.75, 1, 2, 3, 4, 5, 6, 7 or 8.0% conditioning agents, (v) about 0.01, 0.025, 0.05, 0.075, 0.1, 0.2, 0.3, 0.4, 0.5, 0.6, 0.7, 0.8, 0.9, 1, 1.5, 2, 2.5, 3, 3.5, 4, 4.5, 5, 7.5, 10, 12, 15, 17.5 or 20.0% silicones, (vi) about 0.10, 0.2, 0.5, 0.75, 1, 2, 3, 4, 5, 6, 7, 8, 9 or 10.0% mineral oil, (vii) about 0.01, 0.025, 0.05, 0.075, 0.1, 0.2, 0.3, 0.4, 0.5, 0.6, 0.7, 0.8, 0.9, 1, 1.5, 2, 2.5, 3, 3.5, 4, 4.5, 5, 7.5, 10, 12, 15, 17.5 or 20% esters, and/or (viii) a solvent.

The catalyst cream base can comprise a silicone blocker.

Silicones are man-made polymers known for their hydrophobic (water-repellant) characteristics. Silicone can work by forming a thin coating around hair cuticle. The coating or substantivity to the hair causes a delayed or decrease in performance of active agent/agents. Therefore, depending on the amount used it can control the outcome of the process. The terms “silicone” and “silicone blocker” are used herein interchangeably to refer to any silicone-comprising composition that can be used in the present smoothing composition to delay or decrease the potency of the chemicals on the hair. The present smoothing composition can comprise one or more silicone blockers to protect the hair further during the application of the smoothing composition. The use of a silicone blocker can block the full effect of the smoothing compositions such that the hair is not permanently smoothed but is instead semi-permanently smoothed. For example, the smoothing composition can comprise 1, 2, 3, 4, 5 or more silicone blockers.

In one aspect, the smoothing composition can comprise one silicone blocker. Any silicone that can interfere with penetration/restructuring to damage the hair while also providing shine and luster to the hair can be used as a silicone blocker in the smoothing composition. A non-limiting example of silicone blocker is liquid amino phenyl silicone resin (Aminopropyl Phenyl Trimethicone).

For example, the smoothing composition can comprise about 0.01, 0.025, 0.05, 0.075, 0.1, 0.2, 0.3, 0.4, 0.5, 0.6, 0.7, 0.8, 0.9, 1, 1.5, 2, 2.5, 3, 3.5, 4, 4.5, 5, 7.5, 10, 12, 15, 17.5 or 20.0% liquid amino phenyl silicone resin.

In one aspect, the activator composition can comprise about 5%-50% guanidine hydrochloride.

For example, the activator composition can comprise about 5, 10, 15, 20, 25, 30, 35, 40, 45, or 50% guanidine hydrochloride.

In another aspect, the activator composition can comprise about 5%-13% guanidine hydrochloride.

For example, the activator composition can comprise about 5, 6, 7, 8, 9, 10, 11, 12, 13, 14 or 15% guanidine hydrochloride.

In an additional aspect, the activator composition can comprise about 35%-50% guanidine hydrochloride.

For example, the activator composition can comprise about 35, 36, 37, 38, 39, 40, 41, 42, 43, 44, 45, 46, 47, 48, 49 or 50% guanidine hydrochloride.

In an alternative example, amounts of guanidine hydrochloride as described herein can be substituted or combined with a guanidine carbonate activator.

The activator can further comprise a solvent, one or more preservatives, one or more conditioning gums, and one or more buffering solutions.

The activator composition comprises about 30%-85% solvent, about 0.10%-4% preservatives, about 0.05%-5% conditioning gums, and about 0.01%-7% buffering solutions, wherein optionally the solvent is water.

The activator composition can further comprise a blend of active ingredients with cross-linking action based on the synergistic association of unsaturated dicarboxylic acid (itaconic acid), amino acid (arginine), pro-vitamin B5 (D-panthenol), and polysaccharides extracted from linseed and chia seeds, acting through repair and protection mechanisms and proactive treatment of chemically damaged hair.

For example, an activator composition can comprise (i) 30, 40, 45, 50, 55, 60, 65, 70, 75, 80 or 85% solvent, (ii) about 0.10, 0.2, 0.3, 0.4, 0.5, 0.6, 0.7, 0.8, 0.9, 1, 1.5, 2, 2.5, 3, 3.5 or 4% preservatives, (iii) about 0.05, 0.1, 0.2, 0.3, 0.4, 0.5, 0.6, 0.7, 0.8, 0.9, 1, 1.5, 2, 2.35, 3, 3.5, 4, 4.5 or 5% conditioning gums, (iv) about 0.01, 0.025, 0.05, 0.075, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5 or 7% buffering solutions, and/or (v) a solvent.

The catalyst cream base and the activator can be mixed prior to use. For example, they can be mixed about 60, 45, 30, 20, 10, 5, 4, 3, 2, 1 minute or less prior to use.

### Cleansing Shampoo

A cleansing shampoo can comprise about 5%-50% primary and secondary surfactants, about 0.01%-7.0% chelating agent, about 0.01%-5.0% highly charged anionic polymer, and about 0.001%-5.0% buffering agent.

For example, a cleansing shampoo can comprise (i) about 5, 10, 15, 20, 25, 30, 35, 40, 45 or 50% primary and secondary surfactants, (ii) about 0.01, 0.1, 0.5, 0.75, 1, 2, 3, 4, 5, 6, or 7% chelating agent, (iii) about 0.01, 0.025, 0.05, 0.075, 0.1, 0.2, 0.3, 0.4, 0.5, 0.6, 0.7, 0.8, 0.9, 1, 1.5, 2, 2.5, 3, 3.5, 4, 4.5, or 5.0% highly charged anionic polymer, and/or (iv) about 0.001, 0.005, 0.01, 0.025, 0.05, 0.075, 0.1, 0.2, 0.3, 0.4, 0.5, 0.6, 0.7, 0.8, 0.9, 1, 1.5, 2, 2.5, 3, 3.5, 4, 4.5, or 5.0% buffering agent.

A cleansing shampoo can further comprise about 30%-85% solvent and 0.01%-3.0% preservatives, wherein optionally the solvent can be water.

For example, a cleansing shampoo can further comprise (i) about 30, 35, 40, 45, 50, 55, 60, 65, 70, 80 or 85% solvent, (ii) about 0.01, 0.1, 0.5, 0.75, 1, 2, 3, 4, 5, 6, or 7% chelating agent, and/or (iii) about 0.01, 0.025, 0.05, 0.075, 0.1, 0.2, 0.3, 0.4, 0.5, 0.6, 0.7, 0.8, 0.9, 1, 1.5, 2, 2.5 or 3.0% preservatives.

That is, a cleansing shampoo can comprise (i) about 5, 10, 15, 20, 25, 30, 35, 40, 45 or 50% primary and secondary surfactants, (ii) about 0.01, 0.1, 0.5, 0.75, 1, 2, 3, 4, 5, 6, or 7% chelating agent, (iii) about 0.01, 0.025, 0.05, 0.075, 0.1, 0.2, 0.3, 0.4, 0.5, 0.6, 0.7, 0.8, 0.9, 1, 1.5, 2, 2.5, 3, 3.5, 4, 4.5, or 5.0% highly charged anionic polymer, (iv) about 0.001, 0.005, 0.01, 0.025, 0.05, 0.075, 0.1, 0.2, 0.3, 0.4, 0.5, 0.6, 0.7, 0.8, 0.9, 1, 1.5, 2, 2.5, 3, 3.5, 4, 4.5, or 5.0% buffering agent, (v) about 30, 35, 40, 45, 50, 55, 60, 65, 70, 80 or 85% solvent, (vi) about 0.01, 0.1, 0.5, 0.75, 1, 2, 3, 4, 5, 6, or 7% chelating agent, and/or (vii) about 0.01, 0.025, 0.05, 0.075, 0.1, 0.2, 0.3, 0.4, 0.5, 0.6, 0.7, 0.8, 0.9, 1, 1.5, 2, 2.5 or 3.0% preservatives.

### Hair Masque and Serum

A hair serum can be applied after rinsing the hair. Optionally, a serum can be applied after rinsing the hair and applying a hair masque. A hair serum can comprise about 20%-90% solvent, about 0.01%-3% guar hydroxypropyltrimonium chloride, about 0.01%-5% hydroxyethylcellulose, and about 0.10%-8% amodimethicone, optionally wherein the solvent can be water.

For example, a hair serum can comprise (i) about 20, 30, 40, 50, 60, 70, 80 or 90% solvent, (ii) about 0.01, 0.02, 0.05, 0.075, 0.1, 0.2, 0.3, 0.4, 0.5, 0.6, 0.7, 0.8, 0.9, 1.25, 1.5, 1.75, 2, 2.25, 2.5, 2.75 or 3% guar hydroxypropyltrimonium chloride, (iii) about 0.01, 0.02, 0.05, 0.075, 0.1, 0.2, 0.3, 0.4, 0.5, 0.6, 0.7, 0.8, 0.9, 1.25, 1.5, 1.75, 2, 2.25, 2.5, 2.75, 3, 3.25, 3.5, 3.75, 4, 4.25, 4.5, 4.75 or 5% hydroxyethylcellulose, (iv) about 0.10, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5 or 8% amodimethicone, and/or (v) a solvent.

### Laminator Type Serum

In a preferred embodiment, the hair serum can be substituted with a laminator type serum. Laminator is a type of functional conditioning product with smoothing capability and damage prevention. It comprises of multi carboxylic acids (mono, di, and tri-carboxylic acids) capable of straightening, conditioning, and softening effects. Its low molecular size can penetrate deep into the cortex of the hair and bond to keratin. The carboxylic acids are capable of acidic cleavage of the weak bonds that link the polypeptide chains of keratin together and of forming new cross-links.

The laminator type serum composition can comprise various emulsifiers, fatty alcohols, esters, humectants, non-ionic rheology modifiers, bond repair and protective actives, and combinations of carboxylic acids (di or mono or tri) at very low pH.

The smoothing composition catalyst cream base and activator work at high pH and therefore the present technology uses the laminator type serum as a low pH active system to lower the hair pH and create acidic cleavage of the weak bonds, therefore, reducing the damage by restoring the hydrophobic lipid layer and laying down the hair cuticle.

### Kits

Provided herein is a kit comprising: a) a catalyst cream base comprising about 0.10%-10% calcium hydroxide, about 0.10%-5.0% behentrimonium chloride, cetrimonium chloride and/or stearalkonium chloride, about 0.1%-10.0% quaternium-91, about 0.1%-10% emulsifiers, about 0.01%-3.0% guar hydroxypropyltrimonium chloride derivatives, about 0.1%-8.0% conditioning agents, about 0.01%-20.0% silicones, about 0.10%-10.0% mineral oil, about 0.01%-20% esters, and a solvent, wherein optionally the solvent is water, and b) an activator composition comprising about 5%-50% guanidine hydrochloride, about 30%-85% solvent, about 0.10%-4% preservatives, about 0.05%-5% conditioning gums, and about 0.01%-7% buffering solutions, wherein optionally the solvent can be water. The kit can further comprise of chelating cleanser to remove build-up or any other residue left on hair, smoothing emulsion containing calcium hydroxide, and laminator with a blend of different carboxylic acids. It is also envisaged that guanidine hydrochloride can be substituted or combined with a guanidine carbonate activator.

The activator composition can comprise about 35%-50% guanidine hydrochloride. The activator composition can comprise about 5%-13% guanidine hydrochloride. The catalyst cream base can comprise about 0.01% to about 4.5% calcium hydroxide. The silicone blocker can be liquid amino phenyl silicone resin. It is also envisaged that guanidine hydrochloride can be substituted or combined with a guanidine carbonate activator.

Provided herein is a kit for smoothing hair comprising: a) a demineralizing cleansing shampoo composition, b) a catalyst cream base comprising calcium hydroxide, c) an activator composition comprising guanidine hydrochloride, d) a hair serum or laminator type serum, and e) instructions to smooth hair. The kit can further comprise of chelating cleanser to remove build-up or any other residue left on hair, smoothing emulsion containing calcium hydroxide, and laminator with a blend of different carboxylic acids. It is also envisaged that guanidine hydrochloride can be substituted or combined with a guanidine carbonate activator.

The demineralizing cleansing shampoo composition can comprise a primary surfactant, a secondary surfactant, a chelating agent, a highly charged anionic polymer, and a buffering agent. The catalyst cream base can comprise (i) guar hydroxypropyltrimonium chloride or a derivative thereof, (ii) behentrimonium chloride, cetrimonium chloride, and stearalkonium chloride, quaternium-91 and (iii) calcium hydroxide.

The kit can further comprise a silicone blocker. The silicone blocker can be liquid amino phenyl silicone resin.

The compositions and methods are more particularly described below, and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### Example 1

Methods of smoothing hair using a cold process conditioning blend, namely Polyquaternium-37 (anti-static agent and film former), Behentrimonium Chloride (cationic conditioner and frizz control), and Cetearyl Alcohol (fatty alcohol), Stearamidopropyl Dimethylamine (positively charged cationic surfactant and anti-static). When added to a high pH smoothing system, the cold process conditioning blend provides a conditioning ingredient which remains stable throughout the smoothing process. This blend works synergistically before, during, and after the smoothing process with active ingredients in the smoothing system to increase the performance of the smoothing process, and to provide less potential for irritation and over processing (hair damage).

In addition, a silicone blocker (for retardation) can be used. Silicones are man-made polymers known for their hydrophobic (water-repellant) characteristics. Silicone works by forming a thin coating around your hair cuticle. The coating or substantivity to the hair causes a delayed or decrease in performance of active agent/agents. Therefore, depending on the amount used it can control the outcome of the smoothing process. Adding a silicone blocker to the smoothing composition can enhance its performance.

Guanidine hydrochloride is used to ensure less drying effect and smoother frizz free results, which is free or nearly free of formaldehyde. This technology, in one aspect, uses a 5-part system to navigate to the final desired result. It is envisaged that the order of steps is non-limiting and can be rearranged in any order.
Part 1—Cleansing system to remove prior build-up or any other residue left on hair
Part 2—Smoothing emulsion containing calcium hydroxide
Part 3—Activator with guanidine hydrochloride
Part 4—Laminator

### Part 1—Cleansing System:

Primary and secondary surfactants (5%-50%)
Chelating Agent (0.01%-7.0%)
Water (Solvent) (30%-85%)
Preservatives (0.01%-3.0%)
Highly Charged Anionic polymer (0.01%-5.0%)
Buffering Agent (0.001%-5.0%)

### Part 2—Catalyst Cream Base:

May contain emulsifiers (0.1%-10%)
Guar Hydroxypropyltrimonium Chloride Derivatives or Type (0.01%-3.0%)
Behentrimonium Chloride/Cetrimonium Chloride/Stearalkonium Chloride (0.10%-5.0%)
Conditioning Agents (0.1%-8.0%)
Quaternium091 (0.1%-10.0%)
Water (Solvent)
Calcium Hydroxide (0.10%-10%)
Silicones (0.01%-20.0%)
Mineral Oil (0.10%-10.0%)
Esters (0.01%-20%)

### Part 3—Activator

Water (Solvent)—(30%-85%)
Guanidine Hydrochloride (5%-50%)
Preservatives (0.10%-4%)
Conditioning Gums (0.05%-5%)
Buffering Solutions (0.01%-7%)
Unsaturated dicarboxylic acid (itaconic acid)
Amino acid(s) (arginine)
Pro-vitamin B5 (D-panthenol)
Polysaccharides extracted from linseed and chia seeds

### Part 4—Laminator

### Bis-4—PCA Dimethicone

### Disodium PEG-12 Dimethicone Sulfosuccinate

Carboxylic Acids (mono, di, tri)

### Processing of Treatment:

Step 1: Cleanse the hair with cleansing shampoo and towel dry the hair well to remove excess moisture.
Step 2: Mix catalyst cream base with activator for 30 seconds to a minute in a clean color bowl until it becomes a smooth/creamy consistency.
Step 3: Blow-dry hair 100% with tension utilizing a ceramic round brush. Alternatively, hair can be towel dried.
Step 4: Flat iron the hair to achieve a smooth, sleek result. Temperature of the flat iron is 380-450° F. based professional judgment of optimal temperature for the hair treated.
Step 5: Apply mixture from scalp or off scalp to ends in ¼″-½″ sub-sections making sure to saturate the hair from scalp or off scalp to ends.
Step 6: Let the solution process for up to 45 minutes max.
Step 7: Rinse the hair well with chelating cleanser, rinse and towel dry. Next, apply the laminator ½ inch from the scalp to the end and let it sit for about 15 minutes. It is envisaged that laminator processing time is not limited and can be shorter or longer than 15 minutes.
Step 8: Towel dry hair and optionally blow-dry and/or flat iron the hair.

### Example 2

Guanidine hydrochloride is used to ensure less drying effect and smoother frizz free results which is free or nearly free of formaldehyde. This technology uses a 4-part system to navigate to the final desired result. It is envisaged that the order of steps is non-limiting and can be rearranged in any order.
Part 1—Cleansing system to remove prior build-up or any other residue left on hair
Part 2—Smoothing Emulsion containing calcium hydroxide
Part 3—Activator with guanidine hydrochloride
Part 4—Leave-In Serum

### Part 1—Cleansing System:

Primary and secondary surfactants (5%-50%)
Chelating Agent (0.01%-7.0%)
Water (Solvent) (30%-85%)
Preservatives (0.01%-3.0%)
Highly Charged Anionic polymer (0.01%-5.0%)
Buffering Agent (0.001%-5.0%)

### Part 2—Catalyst Cream Base

May contain emulsifiers (0.1%-10%)
Quaternium-91 (0.1%-10.0%)
Guar Hydroxypropyltrimonium Chloride Derivatives or Type (0.01%-3.0%)
Behentrimonium Chloride/Cetrimonium Chloride/Stearalkonium Chloride (0.10%-5.0%)
Conditioning Agents (0.1%-8.0%)
Water (Solvent)
Calcium Hydroxide (0.10%-10%)
Silicones (0.01%-20.0%)
Mineral Oil (0.10%-10.0%)
Esters (0.01%-20%)

### Part 3—Activator

Water (Solvent)—(30%-85%)
Guanidine Hydrochloride (5%-50%)
Preservatives (0.10%-4%)
Conditioning Gums (0.05%-5%)
Buffering Solutions (0.01%-7%)
Unsaturated dicarboxylic acid (itaconic acid)
Amino acid(s) (arginine)
Pro-vitamin B5 (D-panthenol)
Polysaccharides extracted from linseed and chia seeds

### Part 4—Leave-In Serum

Water (Solvent) (20%-90%)
Guar Hydroxypropyltrimonium Chloride Type (0.01%-3%)
Hydroxyethylcellulose Type (0.01%-5%)
Amodimethicone Types (0.10%-8%)

### Processing of Treatment:

Step 1: Cleanse the hair with cleansing shampoo and towel dry the hair well to remove excess moisture.
Step 2: Mix catalyst cream base with activator for 30 seconds to a minute in a clean color bowl until it becomes a smooth/creamy consistency.
Step 3: Apply from scalp or off scalp to ends in ¼″-½″ sub-sections making sure to saturate the hair from scalp or off scalp to ends.
Step 4: Let the solution process for up to 45 minutes max.
Step 5: Rough dry hair 60%-70%. Then blow-dry 100% with tension utilizing a ceramic round brush.
Step 6: Flat iron the hair. 5-10 passes-fewer passes keeps more texture and movement, more passes achieve a flatter, sleeker result. Temperature of the flat iron is defined based professional judgment of optimal temperature for the hair treated. For example, 380-390° F. degrees for thin, fine, chemically processed hair, 400-410° F. degrees for thick, course, single process/virgin hair. For added volume, over direct on root area.
Step 7: After flat ironing, rinse the hair well then wash with the cleansing shampoo and condition with Deep Conditioning Masque.
Step 8: Towel dry hair and apply serum and optionally blow-dry and/or flat iron the hair. Spray from the root to ends and style as desired.

After application of the compositions described herein, following the process described herein, the hair is noticeably smoother and there is a significant reduction in curl and frizz. The application and process also allow for a substantial increase of shine and softness of the hair as compared to the hair prior to application.

While the above examples may be drawn to using a guanidine hydrochloride activator or hair serum, it is also envisaged that guanidine hydrochloride can be substituted or combined with a guanidine carbonate activator and hair serum can be substituted or combined with a laminator type serum.

Although the invention has been described with reference to the above examples, it will be understood that modifications and variations are encompassed within the spirit and scope of the invention. Accordingly, the invention is limited only by the following claims.

1. A method of semi-permanently smoothing hair comprising:
a) contacting hair with a smoothing composition comprising calcium hydroxide, guanidine hydrochloride, and silicone blockers,
b) rough drying the hair with heat, and
c) blow-drying the hair to 100% dryness with tension thereby semi-permanently smoothing the hair.

2. The method of claim 1, wherein contacting comprises contacting hair with a smoothing composition for about ≤45 minutes.

3. The method of claim 1, wherein the tension is created with a ceramic round brush or boar bristle brush.

4. The method of claim 1, wherein contacting the hair with the smoothing composition comprises applying the smoothing composition from scalp to ends in sub-sections and saturating the hair from scalp or off scalp to ends.

5. The method of claim 1, further comprising prior to contacting the hair with the smoothing composition: shampooing the hair with a cleansing shampoo.

6. The method of claim 1, further comprising flat ironing the hair after blow drying.

7. The method of claim 6, further comprising rinsing the hair, washing the hair with a demineralizing cleansing shampoo, and conditioning the hair with a conditioner.

8. The method of claim 7, further comprising towel drying the hair and applying a hair serum or a laminator type serum to the hair.

9. The method of claim 1, wherein the smoothing composition comprises:
(a) a catalyst cream base comprising the calcium hydroxide and a silicone blocker; and
(b) an activator composition comprising the guanidine hydrochloride, wherein the catalyst cream base and the activator are mixed prior to use.

10-28. (canceled)

29. A kit comprising:
a) a catalyst cream base comprising about 0.10%-10% calcium hydroxide, about 0.10%-5.0% behentrimonium chloride, cetrimonium chloride and/or stearalkonium chloride, quaternium-91, about 0.1%-10% emulsifiers, about 0.01%-3.0% guar hydroxypropyltrimonium chloride derivatives, about 0.1%-8.0% conditioning agents, about 0.01%-20.0% silicones, about 0.10%-10.0% mineral oil, about 0.01%-20% esters, and a solvent, wherein optionally the solvent is water, and
b) an activator composition comprising about 5%-50% guanidine hydrochloride, about 30%-85% solvent, about 0.10%-4% preservatives, about 0.05%-5% conditioning gums, and about 0.01%-7% buffering solutions, wherein optionally the solvent is water.

30. The kit of claim 29, wherein the activator composition comprises about 35%-50% guanidine hydrochloride.

31. The kit of claim 29, wherein the activator composition comprises about 5%-13% guanidine hydrochloride.

32. The kit of claim 29, wherein the catalyst cream base comprises about 0.01% to about 4.5% calcium hydroxide.

33. The kit of claim 29, wherein the silicone blocker is liquid amino phenyl silicone resin.

34. The kit of claim 29, wherein the guanidine hydrochloride wherein the guanidine hydrochloride is substituted or combined with guanidine carbonate.

35. A kit for smoothing hair comprising:
a) a demineralizing cleansing shampoo composition,
b) a catalyst cream base comprising calcium hydroxide,
c) an activator composition comprising guanidine hydrochloride,
d) a hair serum or a laminator type serum, and
e) instructions to smooth hair.

36. The kit of claim 35, wherein the cleansing shampoo composition comprises a primary surfactant, a secondary surfactant, a chelating agent, a highly charged anionic polymer, and a buffering agent.

37. The kit of claim 35, wherein the catalyst cream base comprises (i) guar hydroxypropyltrimonium chloride or a derivative thereof, (ii) behentrimonium chloride, cetrimonium chloride, and stearalkonium chloride, quaternium-91, and (iii) calcium hydroxide.

38. The kit of claim 35, further comprising a silicone blocker.

39. The kit of claim 35, wherein the silicone blocker is liquid amino phenyl silicone resin.

40. The kit of claim 35, wherein the guanidine hydrochloride wherein the guanidine hydrochloride is substituted or combined with guanidine carbonate.
